(19) United States
(12) Patent Application Publication
(10) Pub. No.: US 2021/0269848 A1
(43) Pub. Date: Sep. 2, 2021

(54) COMPOSITION OF MICROORGANISMS INDICATING DECONTAMINATION

(71) Applicant: NOVOLYZE, Daix (FR)

(72) Inventors: Laura BOULEY, Dijon (FR); Pablo ALVAREZ, Arc Sur Tille (FR); Karim-Franck KHINOUCHE, Dijon (FR)

(21) Appl. No.: 17/255,120

(22) PCT Filed: Jun. 26, 2019
(86) PCT No.: PCT/EP2019/066959

§ 371 Date: Dec. 22, 2020

(30) Foreign Application Priority Data
Jun. 26, 2018 (FR) FRR1870754

(51) Int. Cl. C12Q 1/22; C12N 1/20; C12Q 1/04; A23C 3/03; A23B 9/02; A23L 3/16

(52) U.S. Cl. CPC C12Q 1/22; C12N 1/205; C12Q 1/04; A23V 2002/00; A23B 9/02; A23L 3/165; A23C 3/03

(57) ABSTRACT

The invention relates to the validation of decontamination processes and in particular to a composition comprising new indicator organisms and a dye, said composition being used for validating the decontamination processes.

### FIELD OF THE INVENTION

The present invention relates to the validation of decontamination processes, and in particular to a composition comprising indicator organisms and a dye, used to validate decontamination processes.

### STATE OF THE ART

Pasteurization processes are applied in many fields, such as the sterilization of equipment and the decontamination of foods, especially dry foods. These processes consist of specific decontamination/sterilization steps or are the concomitant result of a step of a treatment process, such as for example a food cooking step, for example the roasting of products of plant origin.

Products of plant origin, such as almonds or spices, are often contaminated by pathogenic microorganisms present in their growing, storage and use environment which requires a decontamination step before their use for human consumption. This decontamination is often carried out as a temperature treatment of these foods, such as cooking, roasting or drying. However, certain pathogens may be resistant to certain decontamination conditions and care must be taken, before the process is implemented, to ensure that the decontamination objective will indeed be achieved.

This validation cannot be done with a pathogenic microorganism due to the risks of contamination. To this end, so-called “surrogate” indicator microorganisms are used, the behavior of which under treatment conditions must be close to that of the pathogenic organism. Preferably, surrogates will be chosen to be more resistant to the treatment conditions than the pathogens, without behaving too differently from the target pathogens. These surrogates are generally specific to a particular pathogen in a decontamination process, such as for example Enterococcus faecium (ATCC 8459) recommended for the validation of pasteurization processes for almonds likely to be contaminated by pathogenic salmonella. Surrogates are not necessarily the microorganisms phylogenetically closer to the target pathogens, such as for example the genus Citrobacterium, a genus closer to a Salmonella evolutionary pathway which is not described as a surrogate for this pathogen.

New surrogates are described in the application WO 2017/186907, as well as compositions comprising same for use in a decontamination process validation process.

While these surrogates are known, there remains a need to facilitate their use, in particular to better detect their presence and facilitate the counting of microorganisms after the decontamination treatment, in particular by adding a presence marker in the composition of indicator microorganisms.

The use of dyes to detect the presence of microorganisms is known from the state of the art (WO 2008/026104; WO 2009/027855). These are generally dyes that interact with the target microorganisms and mark their presence by the release of a chromophore or a color change. The dyes are generally in a medium suitable for culturing the microorganism so that it can react with them to allow detection of the microorganism by the release or lysis of a chromophore. More particularly, microorganisms and dyes that are degraded by these microorganisms are known in the state of the art (Cheriaa & al., 2012; Olukanni & al., 2013).

For the present invention, it is a matter of using dyes which are “neutral” with respect to the microorganisms under the conditions of use of the surrogates, i.e., these markers must not only resist the decontamination treatment in order to be detected at the end of the process, but also must not affect the viability of the indicator microorganisms both during their formulation and storage and during the decontamination process.

The invention solves this problem by selecting particular dyes that can be added to the composition of indicator microorganisms.

### DISCLOSURE OF THE INVENTION

The present invention relates to an indicator microorganism composition for use in a process for monitoring a decontamination process in which the decontamination process is implemented with said indicator organism.

The composition according to the invention comprises at least one indicator organism and one dye selected from the dyes of the cyclohexadiene-2,5-ylidene family, in particular selected from brilliant blue FCF, patent blue V and brilliant green BS.

In a preferred manner, the dye is brilliant blue FCF (E133). It has been found that this dye not only does not affect the viability of the indicator microorganisms but also increases their resistance to decontamination processes so that they add an extra margin of safety when implementing validation processes.

### DETAILED DESCRIPTION OF THE INVENTION

The present invention relates to an indicator microorganism composition for use in a process for monitoring a decontamination process in which the decontamination process is implemented in the presence of at least one indicator microorganism, or a mixture of indicator microorganisms, and the behavior of said at least one indicator microorganism during said decontamination process is observed.

The composition according to the invention comprises at least one indicator microorganism and one dye of the cyclohexadiene-2,5-ylidene family.

The dyes of the cyclohexadiene-2,5-ylidene family are well known to the person skilled in the art, comprising a common structure of formula

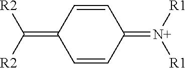

wherein at least one R1 is a C₁-C₆ aliphatic group, in particular ethyl or methyl, and the other is also a C₁-C₆ aliphatic group or an aromatic residue, in particular substituted by at least one sulfonic acid group (—SO₃⁻), and the groups R2 are aromatic residues, of which at least one R2 is an aromatic residue substituted by at least one sulfonic acid group (—SO₃⁻) and at least one R1 is a C₁-C₆ aliphatic group, in particular ethyl or methyl, and the other is also a C₁-C₆ aliphatic group or an aromatic residue, in particular substituted by at least one sulfonic acid group.

Particular mention may be made of
brilliant blue FCF of formula

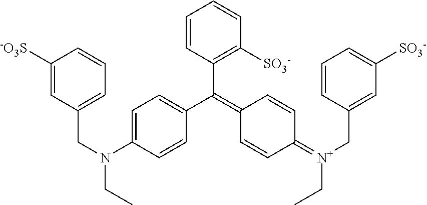

patent blue V of formula

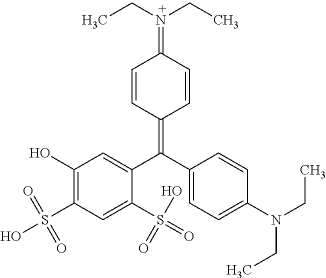

and
brilliant green BS of formula

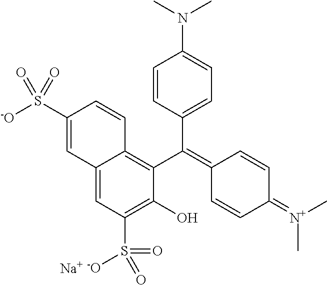

These dyes are known for their use in foods or cosmetics, brilliant blue FCF under the number E133, patent blue V under the number E131 and brilliant green BS under the number E142.

In a preferred manner, the dye is brilliant blue FCF (E133). It has been found that this dye not only does not affect the viability of the indicator microorganisms but also increases their resistance to decontamination processes so that they add an additional margin of safety when implementing validation processes. This improvement in the resistance of the indicator microorganisms is particularly seen from the point of view of their heat resistance, which is a further advantage when the microorganisms are subjected or may be subjected to high temperature conditions, in particular during the manufacture of the compositions or during their transport or storage.

“Microorganism” means a set of several individual microorganisms of the same species. Preferably, the microorganisms are suitable for industrial use, i.e., they can be produced in large amounts by fermentation, up to at least 10⁹ CFU/g, advantageously 10¹⁰ CFU/g, more preferentially up to at least 10¹¹ CFU/g.

Particular mention will be made of the bacteria selected from the species Enterococcus faecium, Geobacillus stearothermophilus, Clostridium sporogenes, Staphylococcus carnosus, Enterobacter hormaechei, Erwinia persicina, Pantoea agglomerans, Pantoea calida, Pantoea dispersa and Pantoea gaviniae, non-pathogenic, preferably capable of being produced industrially, more particularly selected from the following species, Enterococcus faecium, Enterobacter hormaechei, Erwinia persicina, Pantoea agglomerans, Pantoea calida, and Pantoea gaviniae and in particular the strains Enterococcus faecium (ATCC 8459) and the strains deposited at the CNCM under the Budapest Treaty: Enterobacter hormaechei CNCM I-5058, Pantoea agglomerans CNCM I-5059, Pantoea calida CNCM I-5061, Erwinia persicina CNCM I-5062, Erwinia persicina CNCM I-5063, Pantoea agglomerans CNCM I-5054, Pantoea agglomerans CNCM I-5055, Pantoea calida CNCM I-5056.

Other microorganisms used as indicators in the state of the art may also be used in the composition according to the invention. Particular mention will be made of Geobacillus stearothermophilus ATCC 12980 (Okelo et al., 2006 and 2008), Enterococcus faecium NRRL B-2354 (Annous & Kozempel, 1998; ABC, 2007; Bianchit, 2014), Pantoea agglomerans SPS 2F-1 (ABC, 2007), Pantoea dispersa (Fudge & al., 2016), Pediococcus spp. and Pediococcus acidilactici (Borowski et al., 2009; Williams, 2010; Ceylan and Bautista, 2015), Staphylococcus carnosus CS-299 (Vasan et al, 2014), Clostridium sporogenes PA3679, PA3676, and PA3678 (Wallace et al. 2006); Listeria innocua (Sommers et al., 2008), Escherichia coli K12 (Rodriguez et al., 2006), and other E. coli (Gurtler, 2010; Garcia Hernandez et al., 2015).

These non-pathogenic indicator microorganisms have the advantage of showing greater resistance to the implementation conditions of various decontamination processes than that of at least one target pathogenic organism. These target pathogenic organisms are microorganisms responsible for contaminations, in particular pathogenic bacteria of the genera Salmonella, Escherichia, Bacillus, Listeria, Campylobacter, Cronobacter, etc. The purpose of the decontamination process being monitored is to remove all these pathogens in the event they are present in the treated product.

Advantageously, the indicator microorganisms have a higher heat resistance than the target pathogen.

In the decontamination process, the indicator microorganism will be used in a suitable form, corresponding to the form of the target pathogen likely to be present in the product to be decontaminated, in particular in vegetative and/or dry vegetative form.

“Dry form” or “dry vegetative form” means vegetative bacteria which have undergone a drying process allowing their preservation for a determined period of time without modification of their resistance characteristics.

The indicator microorganisms produced by fermentation are then dried for preservation according to techniques known to the person skilled in the art, such as freeze-drying, spraying or drying.

Advantageously, the weight ratio of indicator microorganisms to dye ranges from 0.01 to 5, preferably 1 to 3.

Preferably, the composition according to the invention comprises, in addition to the indicator microorganism and the dye, a suitable support, well known to the skilled person, preferably inert, for example with cryoprotectants such as maltodextrin and/or milk powder and solid supports such as talc, silica and/or activated carbon, and mixtures thereof in any proportion. The support is said to be “inert” in the sense that it does not interact with the metabolism of the bacteria in dry form allowing optimal preservation over time.

The use of a suitable support allows a standardization in the use of microorganisms on different matrices by providing at the same time a better stability of the microorganisms and avoids having to validate the stability of each indicator microorganism on each support after inoculation. It facilitates the implementation of the process according to the invention.

The composition according to the invention is advantageously in a powdered form comprising the mixture of microorganisms in dry form, dye and inert support.

The composition advantageously comprises a content of indicator microorganisms of at least 10¹⁰ CFU/g dry composition.

The composition according to the invention advantageously comprises
from 1 to 5% by weight of dye
from 85 to 98% by weight of inert support and
from 1 to 10% by weight of indicator microorganisms in dry form.

The dry composition is advantageously a powder that has a water activity equal to or less than 0.3.

The composition according to the invention may comprise other additives known to the skilled person, such as activated carbon.

Preferentially, the composition according to the invention consists of the indicator microorganisms in dry form, the dye and the inert support.

The effect of increasing the heat resistance of the indicator microorganisms observed by the addition of dye according to the invention is even more marked for compositions comprising an inert support, in particular when this support is a maltodextrin.

These compositions are prepared according to methods known to the skilled person by mixing according to the usual techniques, the indicator microorganisms in dry form with the support, in the desired proportions, the dye being added before, during or after the mixing of the microorganisms with the inert support. According to another embodiment, the indicator microorganisms are mixed with the suitable support and the mixture is dried for preservation.

Generally, the compositions of indicator microorganisms with the dye and their support are added to the products to be decontaminated in suitable amounts to allow verification of the effectiveness of the decontamination process.

Decontamination processes generally comprise one or more steps of pasteurization, drying, extrusion, roasting, cooking, sterilization, autoclaving and steam treatments.

These processes are well known to the skilled person, in particular pasteurization, drying, extrusion, roasting, cooking, sterilization, autoclaving, steam treatments, pulsed light, high-pressure treatments, or irradiation, gas sterilization (EtO, ppo, ozone) and by disinfectants (bleach, peracetic acid, etc.), in particular for the treatment of natural or manufactured products, such as nuts, aromatic herbs, seeds, spices, food powders, pet food and livestock feed, cereals, etc.

The compositions comprising the surrogates, and mixtures of surrogates, according to the invention may be used, depending on the foods and processes selected, to validate the decontamination of pathogens such as Salmonella, Escherichia coli, Bacillus, Listeria, Campylobacter, Cronobacter sakazakii, etc.

The microorganisms and their support with dye may, if need be, undergo a treatment prior to decontamination, similar to that undergone by the product to be decontaminated, i.e., which will mimic the known processes of product contamination. For example, in the case of natural products that are ground (spices in particular) it is possible to grind them after adding indicator microorganisms on their support to arrive at powders by recreating the classic contamination conditions for natural products.

The monitoring process according to the invention may be implemented before any implementation of a decontamination process on the product to be decontaminated, in order to validate the effectiveness of the decontamination process (validation process). It may also be implemented during decontamination operations on the product to be decontaminated, as a decontamination indicator or as an indicator of conformity of the implementation of the decontamination process (monitoring process).

The process according to the invention, whether it is a validation or a monitoring process, can be implemented under the responsibility of the person carrying out the decontamination or under that of a monitoring or approval body.

The process according to the invention may comprise the steps of
preparing a decontamination monitoring product, said monitoring product comprising a product to be decontaminated associated with an indicator microorganism composition according to the invention,
implementing the decontamination process on the decontamination monitoring product, then
monitoring the presence of viable individuals, during the decontamination process and/or at its conclusion.

The invention also relates to a decontamination monitoring product which comprises a product to be decontaminated associated with an indicator microorganism composition according to the invention.

Depending on the nature and composition of the product to be decontaminated, the composition according to the invention may be deposited on its surface or mixed with the product to be decontaminated, for example when the product and the composition are in powder form.

The skilled person will know how to prepare such a decontamination monitoring product, for example by direct mixing with powdered products (proportion 1%-10%), resuspension of the dry microorganism in a suitable buffer and mixing with different types of products or direct inoculation and mixing on liquid products.

The monitoring microorganisms will be advantageously supplied in kit form, with their use support and, if need be, a set of instructions.

The observation of the behavior of the indicator microorganism generally consists in monitoring the presence of viable individuals, during the decontamination process and/or at its conclusion.

The methods used are known to the skilled person: colony counting on agar and/or molecular methods such as PCR and/or qRT-PCR, or microorganism detection tests such as immunoassays, for example tests using surface plasmon resonance (SPR) technologies, or phage detection tests.

The dye present in the composition according to the invention facilitates the identification of the presence of the indicator microorganism in the monitoring product and thus the samples to be taken at the end of the decontamination process to monitor the presence or absence of remaining viable indicator microorganisms. The absence of viable indicator microorganisms validates the decontamination process, the presence of viable indicator microorganisms being, conversely, a marker of a non-compliant process.

It should also be noted that the effect of increasing the heat resistance of the indicator microorganisms by adding the dye according to the invention provides additional safety in the validation process.

The invention also relates to a kit for monitoring a decontamination process, characterized in that it comprises at least one of the above microorganisms and a suitable support as defined above for use in the decontamination process, and, if need be, a set of instructions.

### DESCRIPTION OF THE FIGURES

FIG. 1 shows the log loss curves of Enterococcus faecium as a function of the dyes after heat treatment at 100° C.

FIG. 2 shows the log loss curves of Enterococcus faecium with and without the dye brilliant blue FCF after heat treatment at 100° C. on an inert support.

“Log loss” refers to the difference between initial viability and viability at a given time.

### EXAMPLES

### Example 1: Test of Dyes: Chlorophyllin and Brilliant Blue FCF

Method: The Enterococcus faecium strain in dry form was mixed with an inert support (maltodextrin) and with each dye individually. The subsequent mixture is placed in an Eppendorf tube in a dry bath at a temperature of 100° C. Samples were taken at 2, 5 and 10 min and cell counts were performed on TSA (Trypticase Soy Agar) medium for 24 h at 37° C.

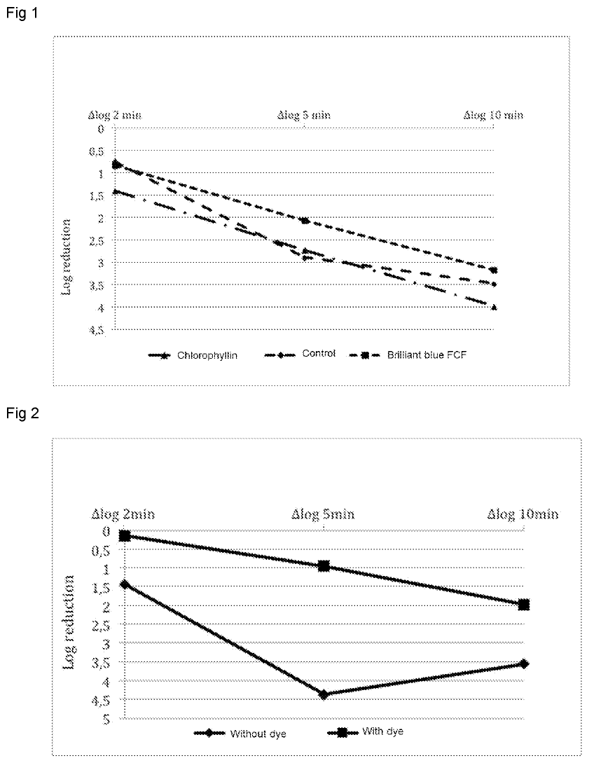

The results are shown in FIG. 1.

### Example 2: Comparison of E. faecium Compositions at 100° C.

The heat resistance of E. faecium with or without the dye brilliant blue FCF is compared in three forms of compositions: fermentation must (Must), without support (Pure) and on inert support (Support).

Must: addition of 3% of blue dye powder to 5 mL of fermentation must in a sterile jar. Vortexing for homogenization, distribution in tubes and heat treatment.

Pure: addition of 3% of dye to 5 g of pure lyophilisate in a sterile jar. Vortexing for homogenization, distribution in tubes and heat treatment.

Support: addition of 1% of pure lyophilisate to 5 g of maltodextrin in a sterile jar (1/100 dilution). Vortexing for homogenization, then addition of 3% of dye. Vortexing then distribution in tubes and heat treatment.

The log loss results in each case are given in Table 1.

TABLE 1

| | | Log loss at 100° C. | | |
|---|---|---|---|---|
| | | 2 | 5 | 10 |
| | | min | min | min |
| Must | With Dye | 1.23 | 1.44 | 2.16 |
| | Without Dye | 1.57 | 2.27 | 4.03 |
| Pure | With Dye | 0.02 | 0.17 | 0.50 |
| | Without Dye | 0.01 | 0.03 | 0.93 |
| Support | With Dye | 0.13 | 0.94 | 1.97 |
| | Without Dye | 0.76 | 2.89 | 3.48 |

The log loss curves for the composition on maltodextrin support, with or without dye, are shown in FIG. 2.

### Example 3: Test on Nonfat Dried Milk NFDM for 3 Strains at 90 and 100° C.

The test is performed for the following 3 strains: E. faecium ATCC 8459, P. agglomerans CNCM I-5055 and A. pascens CNCM I-5181.

Addition of 10% of pure lyophilisate of the strains to 5 g of maltodextrin. Vortexing for homogenization. Addition of 3% brilliant blue FCF dye. Vortexing. Addition of 1% of the dyed strain mixture diluted 1/10 to 5 g NFDM. Vortexing. Deposition of 0.1 g in Eppendorf tubes then heat treatment of the tubes in a dry bath at 90 and 100° C. for 2, 5 and 10 minutes

The results are given in Table 2.

TABLE 2

| | | Log loss at 90° C. | | | Log loss at 100° C. | | |
|---|---|---|---|---|---|---|---|
| | | 2 | 5 | 10 | 2 | 5 | 10 |
| Strain | | min | min | min | min | min | min |
| E. faecium | With Dye | 0.51 | 0.36 | 0.46 | 0.36 | 0.59 | 1.29 |
| ATCC 8459 | Without Dye | 0.4 | 1.65 | 2.65 | 0.11 | 1.6 | 3.1 |
| P. agglomerans | With Dye | 0.07 | 0.07 | 0.37 | 0.13 | 0.67 | 0.72 |
| CNCM I-5055 | Without Dye | 0.68 | 1.28 | 1.18 | 0.54 | 1.32 | 1.53 |
| A. pascens | With Dye | 0.06 | 0.46 | 0.62 | 0.26 | 0.35 | 1.82 |
| CNCM I-5181 | Without Dye | 0.67 | 0.84 | 0.97 | 0.8 | 1.89 | 2.75 |

### Example 4: Test on Black Pepper for 3 Strains at 90 and 100° C.

The test is performed for the following 3 strains: E. faecium ATCC 8459, P. agglomerans CNCM I-5055 and A. pascens CNCM I-5181

Addition of 10% of pure lyophilisate of the strains to 5 g of maltodextrin. Vortexing for homogenization. Addition of 3% of brilliant blue FCF dye. Vortexing. Addition of 1% of the dyed strain mixture diluted 1/10 to 5 g of black pepper followed by a spray of wet fixing agent for good adhesion. Mixing with a sterile spatula. Drying 2 h. Deposition of 0.2 g in Eppendorf tubes then heat treatment of the tubes in a dry bath at 90 and 100° C. for 2, 5 and 10 minutes

The results are given in Table 3.

TABLE 3

| | | Log loss at 90° C. | | | Log loss at 100° C. | | |
|---|---|---|---|---|---|---|---|
| | | 2 | 5 | 10 | 2 | 5 | 10 |
| Strain | | min | min | min | min | min | min |
| E. faecium | With Dye | 0.31 | 0.82 | 1.46 | 1.35 | 1.37 | 2.19 |
| ATCC 8459 | Without Dye | 0.91 | 1.43 | 3.39 | 1.05 | 0.91 | 5.6 |
| P. agglomerans | With Dye | 1.12 | 1.6 | 1.77 | 1.26 | 1.45 | 2.14 |
| CNCM I-5055 | Without Dye | 1.15 | 1.75 | 2.88 | 1.84 | 3.41 | 3.95 |
| A. pascens | With Dye | 0.59 | 0.76 | 0.85 | 0.27 | 0.64 | 0.85 |
| CNCM I-5181 | Without Dye | 0.91 | 1.09 | 1.67 | 1.09 | 1.79 | 2.69 |

### Example 5: Test on Macadamia for 3 Strains at 90 and 100° C.

The test is performed for the following 3 strains: E. faecium ATCC 8459, P. agglomerans CNCM I-5055 and A. pascens CNCM I-5181.

Addition of 10% pure lyophilisate of the strains to 5 g of maltodextrin. Vortexing for homogenization. Addition of 3% of dye. Vortexing. Addition of 1% of the dyed strain mixture diluted 1/10 to 5 g of macadamia. Mixing with a sterile spatula. Drying 2 h. Deposition of 0.3 g in Eppendorf tubes then heat treatment of the tubes in a dry bath at 90 and 100° C. for 0, 2, 5, 10 minutes

The results are given in Table 4.

TABLE 4

| | | Log loss at 90° C. | | | Log loss at 100° C. | | |
|---|---|---|---|---|---|---|---|
| | | 2 | 5 | 10 | 2 | 5 | 10 |
| Strain | | min | min | min | min | min | min |
| E. faecium | With Dye | 0.2 | 0.53 | 1.2 | 0.24 | 0.47 | 1.24 |
| ATCC 8459 | Without Dye | 0.58 | 0.69 | 0.54 | 0.23 | 0.94 | 1.24 |
| P. agglomerans | With Dye | 0.3 | 0.6 | 0.14 | 0.14 | 0.15 | 1.36 |
| CNCM I-5055 | Without Dye | 0.31 | 2.01 | 2.46 | 0.74 | 1.1 | 2.31 |
| A. pascens | With Dye | 0.25 | 0.18 | 0.38 | 0.05 | 0.12 | 0.6 |
| CNCM I-5181 | Without Dye | 0.27 | 0.19 | 1.24 | 0.65 | 0.8 | 1.54 |

### REFERENCES

Annous B A, Kozempel M F. 1998. Influence of growth medium on thermal resistance of Pediococcus sp. NRRL B-2354 (formerly Micrococcus freudenreichii) in liquid foods. J Food Prot. 61(5):578-81.
Bianchini & al., Use of Enterococcus faecium as a Surrogate for Salmonella enterica during Extrusion of a Balanced Carbohydrate-Protein Meal. J. Food Prot., Vol. 77, No. 1
Borowski, A. G., S. C. Ingham, and B. H. Ingham, 2009. Validation of ground- and formed beef jerky processes using commercial lactic acid bacteria starter cultures as pathogen surrogates. Journal of Food Protection 72: 1234-1247
Cheriaa & al., Removal of Triphenylmethane Dyes by Bacterial Consortium, The Scientific World Journal, Vol. 2012, p. 9, 2012.
Enache & al., Development of a Dry Inoculation Method for Thermal Challenge Studies in Low-Moisture Foods by Using Talc as a Carrier for Salmonella and a Surrogate (Enterococcus faecium). Journal of Food Protection, 2015. 78: 1106-1112
Erdogan & al., Evaluating Pediococcus acidilactici and Enterococcus faecium NRRL B-2354 as Thermal Surrogate Microorganisms for Salmonella for In-Plant Validation Studies of Low-Moisture Pet Food Products. Journal of Food Protection, Vol. 78, No. 5, 2015, Pages 934-939.
Fudge & al., The Isolation and Identification of Pantoea dispersa strain JFS as a Non-Pathogenic Surrogate for Salmonella Typhimurium Phage Type 42 in Flour, International Journal of Food Microbiology 219 (2016) 1-6
Garcia-Hernandez R, McMullen L, Gänzle M G. 2015. Development and validation of a surrogate strain cocktail to evaluate bactericidal effects of pressure on verotoxigenic Escherichia coli. Int J Food Microbiol. 205:16-22.
Guidelines for Using Enterococcus faecium NRRL B-2354 as a Surrogate Microorganism in Almond Process Validation. Almond Board of California Guideline, October 2007 (ABC, 2007).
Gurtler & al., Selection of surrogate bacteria in place of E. coli O157:H7 and Salmonella Typhimurium for pulsed electric field treatment of orange juice. International Journal of Food Microbiology 139 (2010) 1-8
Kopit. B. Kim, R. J. Siezen, L. J. Harris, and M. Marco. & al., Safety of the Surrogate Microorganism Enterococcus faecium NRRL B-2354 for Use in Thermal Process Validation, Appl. Environ. Microbiol. 2014, 80(6):1899. DOI:10.1128/AEM.03859-13.
Niebuhr & al., Evaluation of non-pathogenic surrogate bacteria as process validation indicators for Salmonella enteric for selected antimicrobial treatments, cold storage and fermentation in meat, J Food Prot. 2008 April; 71(4):714-8.
Okelo, P. O., D. D. Wagner, L. E. Carr, F. W. Wheaton, L. W. Douglass, S. W. Joseph. 2006. Optimization of extrusion conditions for elimination of mesophilic bacteria during thermal processing of animal feed mash. Animal Feed Science and Technology 129:116-137.
Okelo, P. O., S. W. Joseph, D. D. Wagner, F. W. Wheaton, L. W. Douglass, and L. E. Carr, 2008. Improvements in Reduction of Feed Contamination: An Alternative Monitor of Bacterial Killing During Feed Extrusion. Journal Applied Poultry Research 17: 219-228.
Olukanni & al, Biodegradation of Malachite Green by Extracellular Lacase Producing Bacillus thurigensis RUN1, Journal of Basic & Applied Sciences, 2013, 9, 543-549.
Rodriguez et al., Surrogates for validation of electron beam irradiation of foods, International Journal of FDood Microbiology, 110 (2006) 117-122
Sommers C H, Geveke D J and, Fan X. Inactivation of Listeria Innocua on Frankfurters That Contain Potassium Lactate and Sodium Diacetate by Flash Pasteurization. 2008. J Food Sci 73 (2), M72-M74. 3 2008
Vasan, A., R. Geier, S. C. Ingham, and B. H. Ingham. 2014. Thermal tolerance of 0157 and non-0157 Shiga toxigenic strains of Escherichia coli, Salmonella, and potential pathogen surrogates, in frankfurter batter and ground beef of varying fat levels. Journal of Food Protection. 77:1501-11.
Wallace M, Larson K, Wolf I, Thompson D and Zottola E. Thermal inactivation of Clostridium sporogenes PA 3679 and Bacillus stearothermophilus 1518 in low-acid home-canned foods. 2006 Journal of Food Science 43(6):1738-1740.
Williams, 2010 Williams, P., W. M. Leong, B. H. Ingham, S. C. Ingham, 2010. Lethality of Small-Scale Commercial Dehydrator and Smokehouse/Oven Drying Processes Against Escherichia coli O157:H7-, Salmonella spp.-, Listeria monocytogenes-, and Staphylococcus aureus-inoculated Turkey Jerky and the Ability of a Lactic Acid Bacterium to Serve as a Pathogen Surrogate. Poster presented at the annual meeting of the Institute of Food Technologists. Chicago, Ill. July 2010.
WO 2017/186907, WO 2008/026104, WO 2009/027855

1. An indicator microorganism composition comprising at least one indicator microorganism and one, wherein said dye is selected from the group consisting of the dyes of the cyclohexadiene-2,5-ylidene family.

2. The composition according to claim 1, wherein the dye of the cyclohexadiene-2,5-ylidene family is selected from brilliant blue FCF, patent blue V and brilliant green BS.

3. The composition according to claim 1, wherein the dye is brilliant blue FCF.

4. The composition according to claim 1, wherein the indicator microorganism is selected from the non-pathogenic species Enterococcus faecium, Geobacillus stearothermophilus, Clostridium sporogenes, Staphylococcus carnosus, Enterobacter hormaechei, Erwinia persicina, Pantoea agglomerans, Pantoea calida, Pantoea dispersa and Pantoea gaviniae.

5. The composition according to claim 1, wherein the indicator microorganism is selected from the non-pathogenic species Enterococcus faecium, Enterobacter hormaechei, Erwinia persicina, Pantoea agglomerans, Pantoea calida, and Pantoea gaviniae.

6. The composition according to claim 1, wherein the indicator microorganism is selected from the strains Enterococcus faecium (ATCC 8459) and the strains deposited at the CNCM under the Budapest Treaty: Enterobacter hormaechei CNCM I-5058, Pantoea agglomerans CNCM I-5059, Pantoea calida CNCM I-5061, Erwinia persicina CNCM I-5062, Erwinia persicina CNCM I-5063, Pantoea agglomerans CNCM I-5054, Pantoea agglomerans CNCM I-5055, Pantoea calida CNCM I-5056.

7. The composition according to claim 1, wherein the indicator microorganism is selected from the strains Geobacillus stearothermophilus ATCC 12980, Enterococcus faecium NRRL B-2354, Pantoea agglomerans SPS 2F-1, Pantoea dispersa, Pediococcus spp. and Pediococcus acidilactici, Staphylococcus carnosus CS-299, Clostridium sporogenes PA3679, PA3676 and PA3678, Listeria innocua, and Escherichia coli K12.

8. The composition according to claim 1, wherein the weight ratio of indicator microorganism to dye ranges from 0.01 to 5.

9. The composition according to claim 1, wherein it comprises an inert support.

10. The composition according to claim 9, wherein the inert support is selected from maltodextrin, milk powder, talcum powder, silica, activated carbon and mixtures thereof in any proportion.

11. The composition according to claim 1, wherein the content of indicator microorganisms is at least 10⁹ CFU/g dry composition.

12. The composition according to claim 1, wherein the content of indicator microorganisms is at least 10¹⁰ CFU/g dry composition.

13. The composition according to claim 1, wherein it comprises
from 1 to 5% by weight of dye
from 85 to 98% by weight of inert support and
from 1 to 10% by weight of indicator microorganisms in dry form.

14. The composition according to claim 1, wherein it is a powder which has a water activity equal to or less than 0.3.

15. A process for controlling a decontamination process, wherein said decontamination process is implemented in the presence of at least one indicator microorganism, said decontamination process comprises the step of determining the presence of the at least one indicator microorganism during or at the end of the decontamination process, and wherein the at least one indicator microorganism is comprised in a composition according to claim 1.

16. The process according to claim 15, wherein the decontamination process comprises one or more steps of pasteurization, drying, extrusion, roasting, cooking, sterilization, autoclaving and steam treatments.

17. The process according to claim 16, wherein the decontamination process comprises at least one step of pasteurization, drying, extrusion, roasting, cooking, sterilization, autoclaving, steam treatments, pulsed light, high-pressure treatments, or irradiation, sterilization by gases and by disinfectants.

18. The process according to claim 15, wherein the decontamination process is a process for decontaminating natural or manufactured products.

19. The process according to claim 15, wherein the decontamination process is a process for decontaminating nuts, herbs, seeds, spices, food powders, pet food and livestock feed, or cereals.

20. The process according to claim 15, to validate the decontamination of pathogens selected from Salmonella, Escherichia coli, Bacillus, Listeria, Campylobacter and Cronobacter sakazakii.

21.-24. (canceled)
